(19) United States
(12) Patent Application Publication
(10) Pub. No.: US 2009/0143704 A1
(43) Pub. Date: Jun. 4, 2009

(54) DEVICE FOR MOVEMENT DETECTION, MOVEMENT CORRECTION AND TRAINING

(76) Inventors: Raymond A. Bonneau, Sunnyvale, CA (US); Jeremy Dittmer, Palo Alto, CA (US); Surag S. Mantri, Sunnyvale, CA (US); Tejas N. Mazmudar, Palo Alto, CA (US); Ryan M. McDonell, Sunnyvale, CA (US); Tim F. Ramsey, Seattle, WA (US)

Correspondence Address: DARBY & DARBY P.C., P.O. BOX 770, Church Street Station, New York, NY, 10008-0770, US

(21) Appl. No.: 11/996,121

(22) PCT Filed: Jul. 20, 2006
(86) PCT No.: PCT/US06/28389

§ 371 Date: Jul. 23, 2008

Related U.S. Application Data
(60) Provisional application No. 60/700,909, filed on Jul. 20, 2005.

(51) Int. Cl. A61B 5/11; A61H 1/00

(52) U.S. Cl. 600/595; 601/46

(57) ABSTRACT

According to the present invention, a device (100) that aids in the detection of an unfavorable movement (e g, imbalance) in individuals, such as elderly patients suffering from increased proprioception, includes a body that is constructed to be placed at a targe region of the individual The device (100) includes first and second sensors (200) that are arranged within the body and further include first and second vibrating elements (210) such that when one of the sensors is bent a predetermined extent, a control signal is delivered to the respective vibrating element to cause a vibration thereof for a predetermined time period to alert the individual of the occurrence of an unfavorable movement in the target region before an in ur results.

### CROSS-REFERENCE TO RELATED APPLICATION

The present application claims the benefit of U.S. patent application No. 60/700,909, filed Jul. 20, 2005, which is hereby incorporated by reference in its entirety.

### TECHNICAL FIELD

The present invention relates generally to devices for detecting movement, correcting movement and for training or influencing the action/behavior of an individual, and can be in the form of medical equipment and preventive medical devices or aides that assist in correction of certain behavior and/or movement of the individual, and more particularly, to a device that can be comfortably worn during the day and night to aid in the detection of a certain condition, such as imbalance (e.g., medio-lateral instability) in elderly patients suffering from decreased proprioception.

### BACKGROUND

Improved healthcare has resulted in an aging population in the United States in which the number of people in this country over the age of 65 years is expected to at least double by the year 2035. Unfortunately, as the population ages, more and more people are prone to the health risks and injuries that are commonly associated more with the elderly. For example, falls present a serious risk to the elderly and in this country, one of every three people over the age of 65 will unfortunately experience some type of fall. These falls are the most common cause of injury-related death and the most common cause of injury among all adults over 40 years old in part because of decreasing bone density in elderly people.

Not only is the economic cost of the falls substantial in terms of healthcare costs, etc., but also, the psychological effects of the falls can be substantial. It has been shown that elderly fall victims are prone to neglect, loss of self-esteem and depression. In addition, the falls can interrupt and possibly permanently alter the behavior of the fall victim since a fall victim is less likely to walk and exercise after falls and this further reduces the strength of the patient and increases the odds of a reoccurrence of another fall and subsequent injury.

There are any number of different reasons why a person may fall including but not limited to: dizziness/vertigo, balance or weakness, environment related, confusion, vision, postural hypertension, drop attack, syncope, etc. It should be understood that there is no single reason for a fall by an elderly person but instead any of the above factors or others can trigger or cause a fall. For example, while a number of patients may believe that their fall was an environment related fall, the real cause could be a gait imbalance.

Not only are there a number of different reasons why a person may fall but there are also a number of different mechanisms for falling. In particular, there are five major ways that describe the manner in which an elderly person falls: (1) a forward collapse—knees rigid; (2) a forward collapse—knees flexible; (3) a backward collapse—knees rigid; (4) a sideways collapse—knees rigid; and (5) a fall forward down four stairs—knees rigid. In addition, the impact from these types of falls varies; however, the greatest impact is typically absorbed in the wrist.

Proprioception can be defined as the unconscious perception of movement and spatial orientation arising from stimuli within the body itself. Proprioception and kinesthesia (which is the sensation of joint motion and acceleration) are the sensory feedback mechanisms for motor control and posture. These mechanisms along with the vestibular system are unconsciously used by the brain to provide a constant influx of sensory information. Based on the incoming sensory information, the brain signals for adjustments to be made in the muscles and joints in order to achieve controlled movement as well as to balance the body. Since proprioception is vital to achieve and maintain normal body function, it is often called the body's “sixth sense”.

While there are a number of products (walking aids) on the market to assist the elderly in walking and prevent falls, each of these products suffers from a number of deficiencies and are generally ineffective. For example, there are walker devices that are bulky and too cumbersome for most people and simple canes that are ineffective at preventing falls. A number of systems utilizing accelerometers have been developed but these often suffer from high costs and bulk due to the complex circuitry that is required to produce a working device. Other padded garments and the like are not only uncomfortable and restrictive to wear but also suffer from a lack of compliance.

U.S. patent application publication No. 2004/0173220 discloses a method and apparatus for improving human balance and gait and preventing foot injury by neurological stimulation of the foot and ankle. The stimulation can be provided by electrodes or vibrational actuators, or a combination thereof, with the electrodes and actuators being driven by bias signals generated by a bias signal generator that is coupled to a controller. The signal generator under the control of the controller is adapted to generate a non-deterministic random signal, a repetitive pattern or series of patterns. In other words, the actuators are driven by signal generation circuitry to produce a nondeterministic, noisy, or deterministic (i.e., bias signal) at the surface of the foot. The controller is responsible for controlling the stimulation parameters used to drive the stimulating structures. The controller can be connected to sensing elements which can be pressure sensors that are used to turn off the device, place the device in lower power mode when not in use or detect a swing phase of a limb. The bias signal generator thus drives the device by producing a driving signal that is composed of one or more frequencies to apply neurological stimulation. However, this type of device does not evaluate a rolling action of the ankle when the foot begins to invert or evert and even when, the actuators are passive and actuated by compression, the device has a number of deficiencies in that it is not particularly suited for use in detecting a rolling action of the ankle. As described herein, a rolling action of the ankle is marked by foot movement that is not necessarily an event where compression takes place as described in the '220 publication and therefore, by the time a sensor is compressed according to the '220 arrangement, it may be too late and the ankle may have rolled too much to overcome and prevent the fall from occurring.

In addition, other conditions and disorders exist that can be aided if a person is made more aware of certain movements and/or behaviors. For example, carpal tunnel syndrome is a painful progressive condition caused by compression of a key nerve in the wrist. It occurs when the median nerve, which runs from the forearm into the hand, becomes pressed or squeezed at the wrist. Symptoms usually start gradually, with pain, weakness, or numbness in the hand and wrist, radiating up the arm. As symptoms worsen, people might feel tingling during the day, and decreased grip strength may make it difficult to form a first, grasp small objects, or perform other manual tasks. In some cases no direct cause of the syndrome can be identified. Most likely the disorder is due to a congenital predisposition—the carpal tunnel is simply smaller in some people than in others. However, the risk of developing carpal tunnel syndrome is especially common in those performing assembly line work. One treatment for this disorder involves resting the affected hand and wrist, avoiding activities that may worsen symptoms, and immobilizing the wrist in a splint to avoid further damage from twisting or bending. However, some individuals are not able to recognize when they may be bending the wrist in the wrong direction or at an improper angle which exacerbates the condition and potentially does further damage or lengthens the recovery time.

One other condition that is potentially dangerous and fatal is when a person is overcome by sleep or otherwise begins to lose concentration and focus when driving a vehicle. Typically, as the person is experiencing such a condition, the driver's head will no longer be upright and focused on the road but instead the driver begins to nod down or to the side as the driver becomes progressively sleepier or is otherwise not in total command or control of the situation. It would be desirable to provide some type of device that can immediately alert the driver as well as provide some type of corrective action to assist the driver in regaining control.

### SUMMARY

According to one aspect of the present invention, a device for detecting movement and providing corrective feedback to an individual to influence the movement of the individual includes a first means for detecting movement of the individual and a controller in communication with the first means and configured to receive and store information relating to the detected movement of the individual over a period of time. The controller defines a range of movements that is considered acceptable based on the detected movement and stored information. The device also includes a second means for providing sensory feedback to the individual when the controller determines that the detected movement lies outside of the acceptable range to assist in correcting the movement of the individual by alerting the individual to the occurrence of undesired movement. The feedback is thus administered to the individual based directly on the detected movement.

According to one embodiment of the present invention, a device that aids in the detection of imbalance (e.g., medio-lateral instability or anterior-posterior instability in an ankle/foot region) in individuals, such as the elderly, suffering from decreased proprioception includes an ankle brace that is constructed to be placed at the ankle/foot region of the individual. The device has first and second sensors that are disposed within the ankle brace and first and second vibrating elements such that when one of the sensors is bent a predetermined extent, a control signal is delivered to one of the vibrating elements to cause a vibration thereof for a predetermined time period to alert the individual of either inversion or eversion of the ankle/foot or anterior/posterior instability before a fall results.

According to one exemplary embodiment of the present invention, a device is provided to aid in the prevention of a fall by an individual, such as an elderly patient, and includes an ankle brace that is constructed to be worn around an ankle and foot region of the wearer to provide support thereto.

The device has first and second sensors arranged so that when the device is worn, the first sensor is positioned along one side of the foot, while the second sensor is positioned along the other side of the foot. Each sensor is of the type that when the sensor is bent, the resistance thereof gradually increases and flexion of the sensor is converted into a voltage value.

A microprocessor is operatively connected to the first and second sensors for receiving and sampling current voltage values outputted from the sensors. The microprocessor includes a memory for storing a history of the voltage values outputted from the sensors. In addition, the device includes first and second vibrating elements in communication with the microprocessor. The first vibrating element is located proximate the first sensor, while the second vibrating element is located proximate the second sensor. The microprocessor sends a control signal to operate one of the first and second vibrating elements when a difference between a current voltage value of one sensor and an average of the stored history of voltages values of the one sensor exceeds a predetermined threshold resulting in vibration of the respective vibrating element for a predetermined time period to alert the individual of either inversion or eversion of the foot. The device is of a portable nature and is powered by a power source, such as a plurality of batteries, that is contained in a housing that contains the microprocessor and is positioned along the outer surface of the ankle brace.

According to another aspect of the present invention, a method for detecting imbalance in an individual who is suffering from decreased proprioception prior to the individual falling includes the step of disposing an ankle brace around an ankle and foot region of the individual. The ankle brace includes first and second sensors arranged so that when the device is worn, the first sensor is positioned along one side of the foot, while the second sensor is positioned along the other side of the foot. The ankle brace also has a first vibrating element located proximate the first sensor and a second vibrating element located proximate the second sensor.

The method further includes the step of sensing either inversion or eversion of the foot when one of the first and second sensors bends a predetermined extent due to ankle and foot movement; and instructing a respective one of the first and second vibrating elements to vibrate for a predetermined amount of time depending upon which sensor is bent to alert the individual of either inversion or eversion of the ankle/foot.

Further aspects and features of the exemplary device disclosed herein can be appreciated from the appended Figures and accompanying written description.

### BRIEF DESCRIPTION OF THE DRAWING FIGURES

FIG. 1 is a perspective view of a device for detecting and correcting medio-lateral instability in an ankle/foot region of a person according to one exemplary embodiment of the present invention;

FIG. 2A is a cross-sectional view taken essentially along the line 2A-2A of FIG. 1;

FIG. 2B is a cross-sectional view taken essentially along the line 2B-2B of FIG. 2A;

FIG. 3 is a plan view of a brace body of the device in an open position;

FIG. 4 is a side view of a lateral ankle anatomy illustrating a location of the vibrating element;

FIG. 5 is a diagrammatic view of eversion and inversion of the ankle/foot region along with general movement of the sensors and vibrating elements;

FIG. 6 is a top perspective view of one exemplary sensor;

FIG. 7 is a top perspective view of one exemplary vibrating element;

FIG. 8 is a side view of one exemplary controller;

FIGS. 9A, 9B, 9C illustrate a circuit diagram associated with the working components of the device when matched along their respective match lines; and

FIG. 10 is a flow chart of the operation of software that is associated with the controller.

### DETAILED DESCRIPTION OF PREFERRED EMBODIMENTS

The present invention is directed to a device that detects movement of an individual and provides corrective feedback to the individual in order to assist or train the behavior and/or movement of the individual based directly on the detected movements of the individual. The present invention can be embodied in any number of different forms and can be used for any number of different applications including those described below.

For example, the present invention is illustrated and described with reference to a device that is designed to monitor the movement of an individual's ankle and foot and provide corrective feedback to the individual when the device receives information that indicates that the individual is moving the ankle and foot in an undesired manner. It will be appreciated that the application of a device according to the present invention in the ankle and foot region of the individual to monitor the movement in this region is merely exemplary in nature and a device according to the present invention can be embodied in different forms for application to different locations of the individual's body to monitor movements thereat. Other applications, including the use of the present device at the wrist and head locations to monitor movement thereof are disclosed herein.

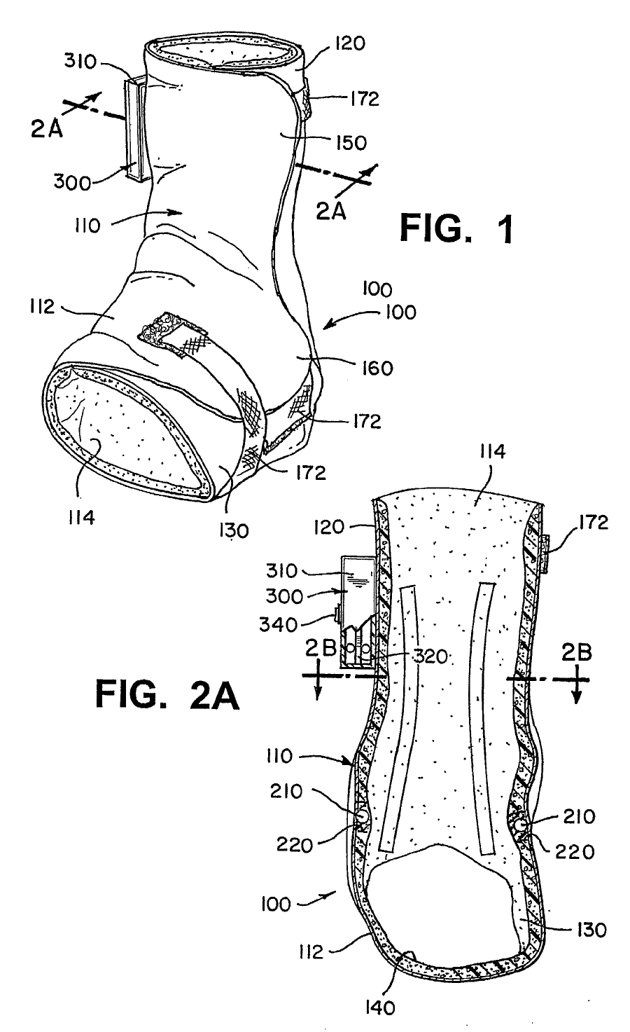

FIGS. 1-3 illustrate a device 100 according to one exemplary embodiment of the present invention which is intended to help prevent falls among individuals, such as elderly patients, and more particularly, the device 100 is constructed to be comfortably worn during the entire day and night as an aid in the detection of imbalance in an elderly patient suffering from decreased proprioception. In general, the device 100 is in the form of an electromechanical system that senses potential imbalance in the individual, then sends a vibratory message to the individual (wearer) to correct for it.

It will be understood that the device 100 is generally a device for detecting movement, for correcting movement and for training an individual how to perform a task, such as walking with increased stability. However, the present invention is not limited to being used with elderly patients since other patients experiencing the above instability can benefit from the present invention.

As illustrated in FIGS. 1-3, the device 100 can in one embodiment take the form of an ankle brace that incorporates a number of features described in detail below to detect and inform the user of medio-lateral instability so that the user can correct his or her balance and avoid a potentially serious fall that could lead to injury or even death. The device 100 is configured so that when the individual's ankle begins to roll and is detected, the sensory and operative parts of the device 100 immediately alert the user of such ankle roll and prompt the user to correct the roll. The device 100 therefore generally functions by detecting ankle roll and is configured in view of the various features of the human anatomy, namely, the foot and ankle. The foot alone contains 26 bones and 57 joints and plays a key roll in weight bearing and shock absorption. What is thought of as the ankle is actually a group of bones and joints that include parts of both the foot and the leg. The dominant modes of motion in the ankle are those of dorsi-flexion (lifting the toes) and plantar flexion (pointing the toes). The true ankle joint is the joint for enabling this motion and is found at the interface between the lower leg bones (the tibia and fibula) and the talus in the foot. Though critical to basic foot motion, the movement in the ankle joint does not account for ankle roll, but instead, the ankle roll occurs in the lower subtalar joint (below the talus) as well as in the transverse tarsal joints. Motion in the subtalar joint allows for inversion and eversion of the heel, and motion in the transverse tarsal joints permits corresponding supination and pronation of the forefoot.

There is a link between a change in ankle position and proprioception and age and in particular, as people age, there is a decreased ability to sense a change in ankle position and thus, an individual's ankle may begin to roll and unfortunately, the person will not realize it. The threshold for awareness of ankle inversion in substantially greater for the elderly compared to younger people. This is reflected in that the degree of roll (inversion) that is detectable in younger persons can be on the order of about 0.06°, while the degree of roll detectable in the elderly can be on the order of about 0.35° (the maximum ankle inversion is on the order of about 3°). Thus, there appears to be a link between elderly loss of proprioception with the inability to sense and react to both plantar and ankle forces and in general, there is a link between a person's imbalance and decreased proprioception below the ankle.

The device 100 includes a main body (brace) 110 that is to be worn by the user about the ankle and extend also along the user's foot and lower leg as shown. The main brace body 110 is formed of a flexible, readily bendable material, and can be formed from synthetic materials (polymeric materials) or can be formed of natural materials (cotton, etc.) or a combination thereof. For example, the brace body 110 can be formed of synthetic materials, such as nylons, etc., and in one embodiment, the brace body 110 is formed of a neoprene material.

The brace body 110 can be unfolded and laid flat on a surface and is cut according to a predetermine pattern to define a number of structural and functional features. As shown in FIGS. 1-3, the brace body 110 includes an outer surface 112 and an opposing inner surface 114, as well as an upper portion 120 to be secured to the user's lower leg above the ankle and a lower portion 130 that is to be secured to the user's foot. The inner surface 114 can be formed of a softer layer of material since it is in contact with the user's body, while the outer surface 112 can include a more rigid material since it is not in contact with the user and may be exposed to the elements and the environment.

The brace body 110 is preferably symmetrical about an axis A-A and includes a central slot 140 formed primarily in the lower portion 130. As shown, the central slot 140 is formed along the axis A-A and is oriented longitudinally and can come in any number of different shapes and sizes so long as a heel of the user can be received through the central slot 140 when the brace body 110 is worn on user's foot. In the illustrated embodiment, the central slot 140 has an oblong like shape; however, other shapes can be used so long as it serves as a heel slot that serves to locate and help in the attachment of the brace body 110 to the user.

The width of the brace body 110 is slighter greater across the lower portion 130 and the upper portion 120 is in the form of a first pair of wings (wing-like structures) 150 formed on opposite sides of axis A-A and the lower portion 130 is in the form of a second pair of wings (wing-like structures) 160, with a pair of arched cut outs 170 being formed between the upper portion 120 and the lower portion 130 so as to define the first and second pair of wings 150, 160. It will be appreciated that the first pair of wings 150 is folded over and attached to one another above the ankle, while the second pair of wings 160 is folded over and attached to one another below the ankle and more particularly across the foot.

The slot 140 can be reinforced along its peripheral edge by having extra stitching or the like that serves to reinforce and prevent any tearing along the peripheral edge of the slot 140.

Any number of different fastening and tightening means can be used to securely attach the brace body 110 to the user's ankle and foot. For example, ratcheting elements, snap-fit members, buttons, etc., can be used and as illustrated, hook and loop type fasteners 172 can be used to securely attach both the upper portion 120 and the lower portion 130. In other words, a first set of hook and loop type fasteners 172 is attached to the first pair of wings 150 for securely tightening the upper portion 120 and a second set of hook and loop type fasteners 172 is attached to the second pair of wings 160. The brace body 110 is attached to the user's ankle and foot by first positioning the inner surface 114 face up and then inserting the heel of the foot through the central slot 140 and then attaching the upper portion 120 and the lower portion 130 by folding the respective pair of wings 150, 160 over one another and then tightening the respective hook and loop type fasteners 172.

One exemplary brace body 110 is commercially available under the product name AthleticWorks™ and is in the form of a neoprene ankle brace with hook and loop type straps for tightening. This type of brace allows one size to fit a much larger range of users than a standard neoprene or lace up brace. The weight of the brace body 110 can be about 200 grams; however, this is merely illustrative.

The brace device 100 includes a number of working components that are associated therewith to provide the desired response in the wearer when an ankle roll is detected. In particular, the inner surface 114 of the brace body 110 includes a pair of sensors 200 for sensing the ankle roll and a pair of vibrating elements 210 associated therewith for alerting the user at the onset of an ankle roll.

In order to accommodate the pair of sensors 200 and the vibrating elements 210, slots or recessed channels 220 are formed in the inner surface at specific locations where the elements are to be placed. After insertion of the members in the slots or recessed channels 220, the members can be covered with a layer of material (e.g., a 1/32^{nd} of an inch of neoprene material) so as to imbed the members and improve durability and comfort of the brace body 110.

The sensors 200 are designed to detect the rolling action of the ankle both in terms of an inversion of the foot and an eversion of the foot and therefore, the sensors 200 are configured to be only sensitive in bending in one direction and therefore, a pair of sensors 200 is required for each foot in order to detect both inversion and eversion motion. The recessed channels 220 are formed in the inner surface 114 so as to position one sensor 200 on the medial side of the foot near the medial malleoli, while the other sensor 200 is placed on the lateral side of the foot near the lateral malleoli. The lateral and medial malleoli are protuberances on the lower ends of the tibia and fibula. The sensors 200 extend roughly from the malleoli to the plantar surface of the foot, thereby permitting the sensors 200 to bend as much as possible when an ankle roll occurs so as to be able to detect such event. It will also be appreciated that the sensors 200 can be disposed along the inner surface 114 without need for the channels 220.

There are a number of different types of sensors 200 that can be used in the brace device 100 of the present for detecting an ankle roll condition. The sensors 200 described below differ in terms of their sensitivity, durability and size, as well as the ease of attachment of the sensors 200. One type of sensor 200 is commercially available under the name Flexiforce sensor and is constructed of two layers of substrate (polyester/polyimide) film that provides a resistance that varies with applied load and provide a change in resistance of about 10% when the ankle is at its maximum inversion. The Flexiforce sensors are durable but their sensitivity is very reliant on the placement of the sensors and this type of sensor is better suited for direct loading in contrast to detecting an ankle roll. One other type of sensor is known as a stretch sensor that is a flexible cylindrical cord with spade electrical fixings at each end. This type of sensor acts as a variable resistor and provides a change in resistance of about 15% when the ankle is at its maximum inversion. While being soft and compliant, this type of sensor can pose some difficulties in mounting the sensors within the device 100. Yet another type of sensor is a piezo film sensor that produces a voltage in response to bend, acceleration, and vibration. This type of sensor is very sensitive and gives a large output for a given bend or vibration input; however, their heightened sensitivity makes their use in the present invention difficult since the impact associated with walking is difficult to distinguish from an ankle roll event.

As illustrated in FIG. 6, the sensors 200 are preferably in the form of flex sensors that are elongated piezo-resistive strips 202 that provide and maintain a change in resistance based on a bending action (extent of the bend). The sensitivity of the strips 202 is similar to the above described stretch and flexiforce sensors in that it is about 10% at maximum ankle inversion. The ability to hold this change is resistance steady, in addition to the relative ease of which they the sensor strips 202 can be attached and the thin, flexible construction of the strips 202, make the sensor strips 202 very suitable for use in the present invention for detecting ankle roll. At one end of the strips 202, electrical contacts are provided to permit the strips 202 to be electrically attached to other conductive members, such as electrical wires.

The strips 202 are disposed within the pair of elongated recessed channels 220 that are formed in the inner surface of the brace body 110 in the medial or lateral side of the foot near the medial and lateral malleoli. A material, such as the same neoprene material of the brace body 110, is preferably used to cover the buried strips 202 and provide a more uniform, comfortable inner surface 114.

However, it will be understood that a number of different types of sensors can be used so long as they are configured to detect movement of the person and once the degree of movement exceeds some threshold, a signal from the sensor is sent to the controller for initiating some type of activation or providing feedback to the user as described below.

The device 100 includes a pair of vibrating element 210 (actuating elements or feedback elements) and similar to the pair of sensors 200, the vibrating elements 210 are located on the medial and lateral side of the foot. However, it will be appreciated that the placements of both the sensors 200 and the vibrating elements 210 along the medial and lateral sides of the foot are merely one application and both of these elements 200, 210 can be placed in another locations, including posterior/anterior locations or some other locations.

Human response to vibratory input, such as that produced by the vibrating elements 210 as described in more detail below, varies with the frequency of vibration, location and amplitude of vibration input. Human skin is most sensitive to vibrations with frequencies of approximately 250 Hz. According to one embodiment and when the vibrating elements 210 are in the form of vibrating motors 210 which are run at a voltage such that the maximum frequency approaches about 200 Hz. This means that the vibratory stimulus with ankle roll allows feedback near the maximum level of sensitivity. In one example, the motors 210 were tested at a rated voltage at 1.3 V of 140 Hz; however, it will be appreciated that the motors 210 will be run at a higher voltage and therefore, the frequency will increase.

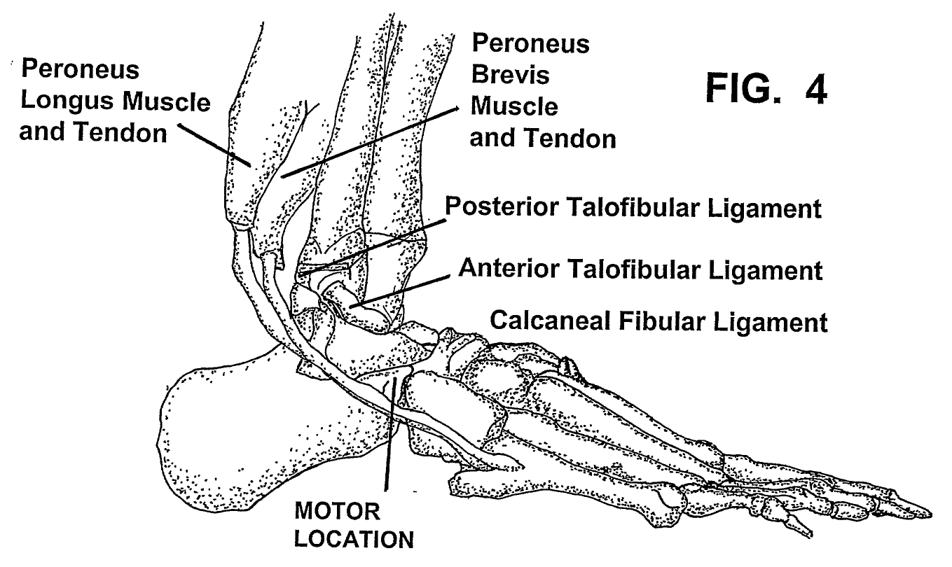

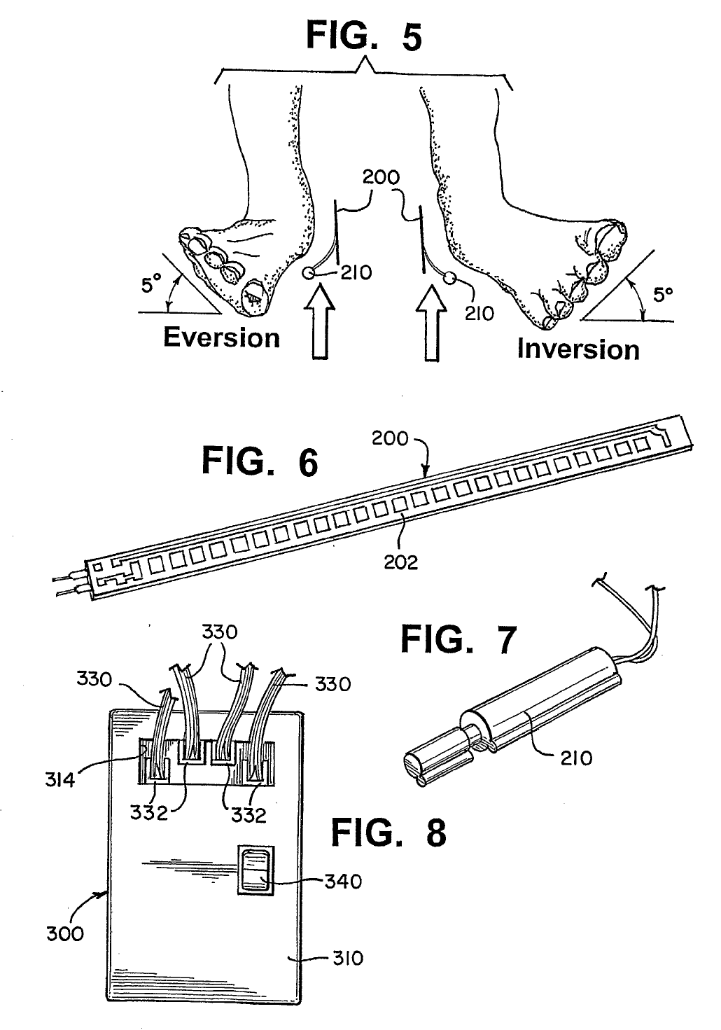

The quickest reaction time to a vibratory input, such as operation of the vibrating motors 210, occurs when the stimulation is applied near a tendon or muscle bunch. This can be attributed to the fact that muscles consist of mechanoreceptors. In view of this information and as illustrated in FIG. 4, the vibrating motor 210 is preferably positioned directly above the calcaneal fibular ligament underneath the lateral malleolus. This ligament is a major ligament in the foot and at this point, there are several other ligaments in the vicinity that can also detect the vibration when one of the vibrating motors 210 is operated. Preferably, the vibrating motors 210 are placed in the same locations with respective to their respective feet. In addition, this placement of the vibrating motor 210 close to the respective sensor 200 minimizes the wiring hardware that is used to connect the two together.

The vibrating elements 210 can be several different types of vibrating elements so long as they are intended and suitable for use in the present application. For example, the vibrating elements 210 can be in the form of vibrating motors that when activated vibrate. In one embodiment, the vibrating elements 210 are in the form of motors similar to those found in cell phones and pagers and run at 3 V at approximately 25 mA. The advantage of this type of motor is that the current drawn is attractive for the intended use; however, the vibration of this type of motor is relatively low at about 75 Hz. Unfortunately, this level of frequency is below the frequency at which human skin is most sensitive to vibration. In addition, the frequency is essentially constant making it difficult to achieve varying levels of vibratory stimulation. Moreover, it can be difficult to localize the vibration with the type of motor and in the device 100, a localized vibration is desired for effective proprioception enhancement.

In view of the foregoing, preferably, the vibrating elements 210 are of the type that have an eccentric mass on an output shaft and draw approximately 50 mA at 1.3 V as illustrated in FIG. 7. It will be appreciated that the power requirement for this type of motor is greater than other types of motors (such as cell phone or pager motors), this type of motor includes a frequency of vibration from about 140 Hz to about 200 Hz, which is much closer to the maximum sensitivity of humans, which is about 250 Hz as previously mentioned. Advantageously, the vibration is highly localized and pulse width modulation can potentially be used to vary the frequency of the vibration feedback.

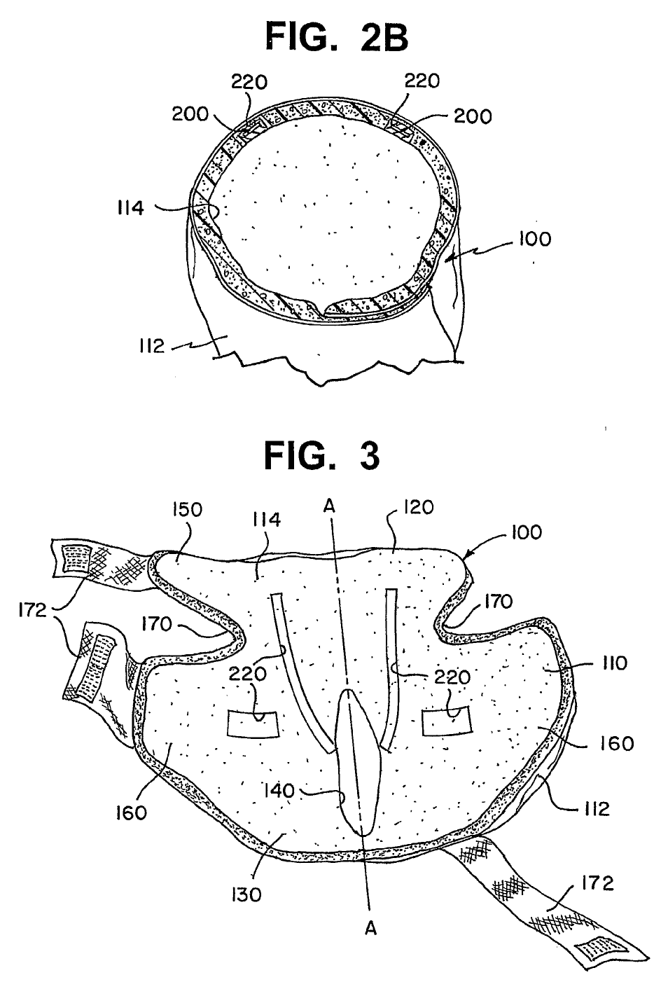

The device 100 also includes a controller 300 that controls the operation of the working components of the device 100 and is in communication with the sensors 200 and the vibrating elements 210. As shown in FIGS. 1-2, the controller 300 is disposed on the outer surface 112 of the body 100, while the other components are on the inner surface 114 since these components must interact with and be responsive to movement of the foot and thus are placed next to the skin. In contrast, the controller 300 is located on the outer surface 112 of the body 110 at a location that minimizes and interference with the wearer and maximizes the comfort of the wearer.

As shown in FIGS. 1, 2 and 8, the controller 300 includes a housing 310 that contains a printed circuit board 320 or the like as well as electrical wiring 330 that serves to electrically connect the printed circuit board 320 to the working components, such as the sensors 200 and vibrating elements 210. When the housing 310 contains a PCB/processor 320, the housing 310 can be thought of as a circuit box that can be formed of two parts that are openable and closeable relative to one another so as to expose the inside of the housing 310 where the PCB 320 is located.

The electrical wiring 330 can be done with any number of different materials and structures. For example, the electrical wiring 330 can be 30 gauge redundant ribbon cabling to minimize size while keeping the resistance in the wiring 330 low. The wiring 330 runs along the outer surface 112 of the brace body 110. In addition, the wiring 330 is covered with a strip of material, such as neoprene material that is 1/32^{nd} of an inch thick, etc., to keep the bulk down while hiding the wiring 330.

When the wiring 330 is in the form of ribbon cables, the cables preferably having Molex type connectors 332 or other types of connectors that can be easily plugged and unplugged from the housing 310 containing the circuitry (PCB 320). In other words, the housing 310 can contain a slot or window 314 through which one set of the connectors 332 is accessible and connected to the PCB 320. In addition, a switch 340 is provided for turning the device 100 on or off. The switch 340 is accessible through an opening or window 342 formed in the housing 310 to permit the user to turn the device 100 on or off by simply manipulating the switch 340 as by sliding, rotating, or pressing the switch 340. If the device 100 is removed from the wearer's body, then the device 100 should be turned off to preserve the battery charge.

The PCB 320 of the controller 300 is preferably in the form of a programmable integrated circuit (PIC) microprocessor in which an algorithm resides for operating the device 100 in the manner described below. In one embodiment, the microprocessor 320 is a PIC 16F88 from Microchip that offers the following features: 10 bit analog to digital ports (this feature allows the detection of small changes in the sensors 200); onboard multi-speed (8 MHz to 31.25 KHz) precision internal oscillator; can be programmed in low voltage programming mode; it is inexpensive; C code can be ported into assembly using free C compilers; and ME218 lab is equipped with software (EPIC) to upload the code for PIC processors. However, other controllers can equally be used and the above microprocessor is merely on example.

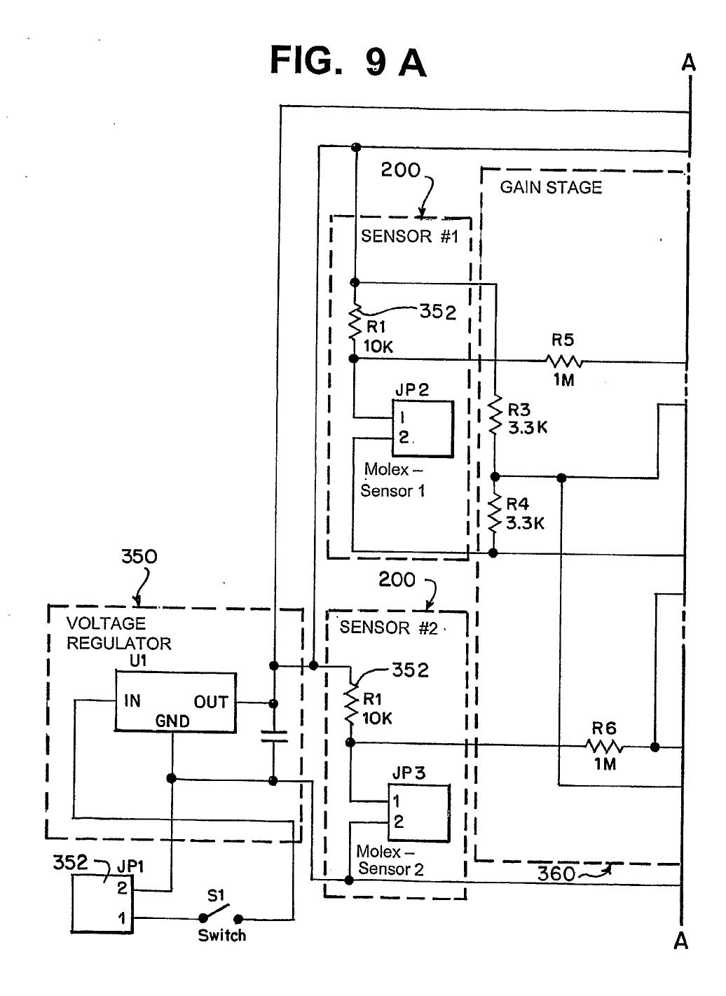

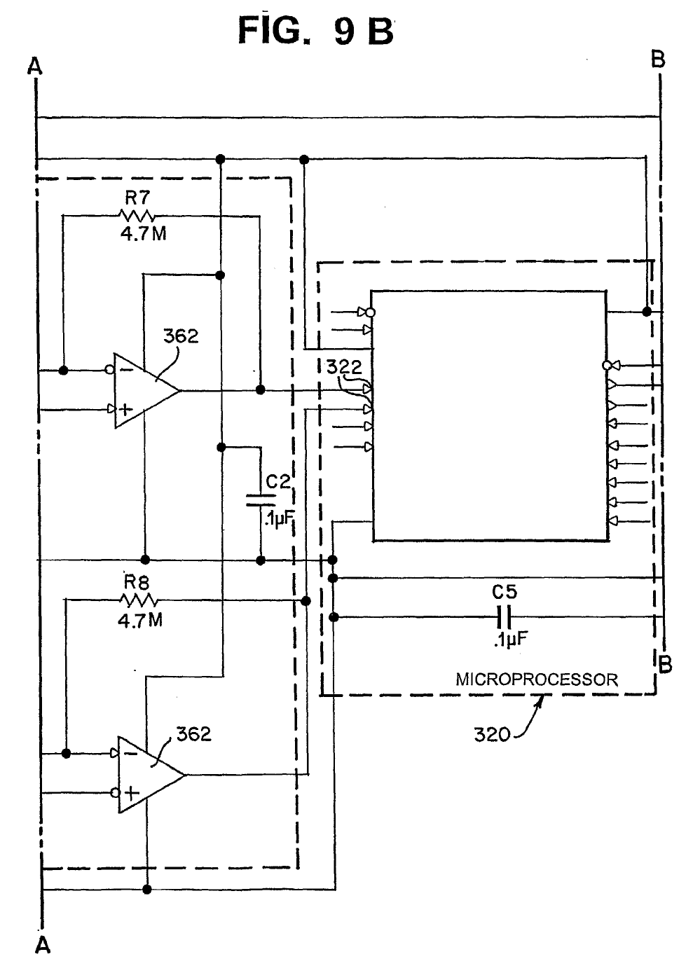

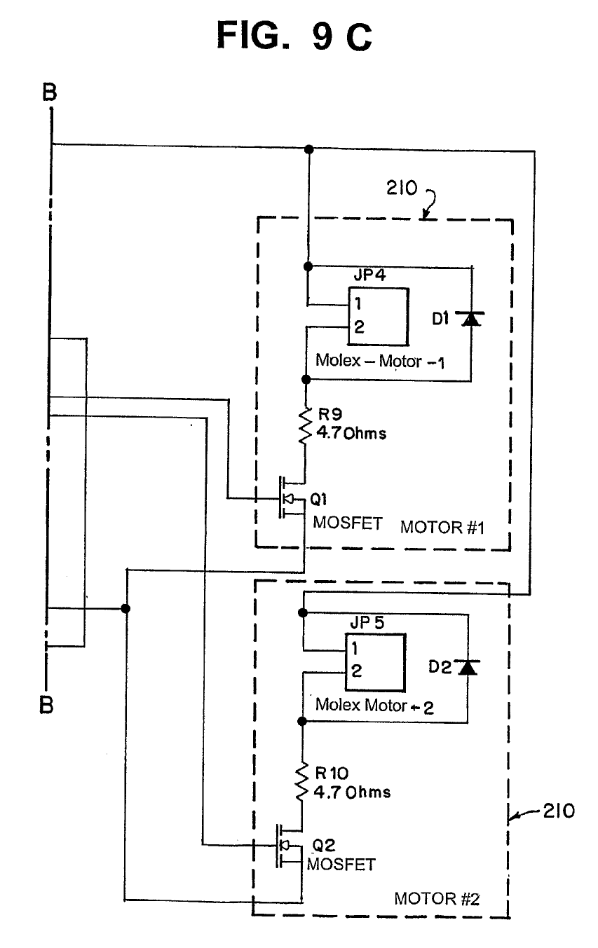

One exemplary controller 300 includes five main components including a voltage regulator 350, two sensors 200, a gain stage 360, the microprocessor 320 and a pair of motors (vibrating elements) 210. FIGS. 9A, 9B, and 9c represent a schematic of one exemplary circuit for use in the device 100.

The voltage regulator 350 is constructed so that it down regulates an input that is over 5 volts to 5 volts. In other words, the regulator 350 will take input voltages greater than 5 volts and then down regulate the voltage to 5 volts. This permits several batteries to be placed in series, while consistently maintaining 5 volts and ground on the power rails associated with the device 100 and in particular, the electronics thereof. In other words, the output of the power source (e.g., batteries) can be greater than 5 V; however, when this voltage is input into the regulator 350, it is regulated down to 5 V to ensure proper operation of the device 100.

As previously mentioned, one component of the electronics of the device 100 is the sensors 200 that have the unique property of changing resistance when they are bent. When the sensor 200 is in a position of no flexion, it has a nominal resistance of about 10K ohms. However, due to existing natural bends, the resistance of the sensor 200 is about 12K when in a position of no flexion. As the sensor 200 is bent, the resistance gradually increases. When the sensor 200 is bent at 90 degrees, the resistance of the sensor 200 ranges from about 30-40K ohms. The flexion of the sensor 200 is converted into a voltage using a simple voltage divider setup. A 10K resistor 352 is placed in series with the sensors 200 that have a resistance of about 12K. The point between the two resistors hovers about 2.7 volts with no flexion and is maximally about 4 volts at full flexion.

The gain stage 360 is an inverting operational amplifier (gain) stage that is set up to have a gain of 7.7 and a reference voltage of 3.0 V. Consequently, when the sensor 200 is not in flexion, the output is a full 5 V. When the sensor 200 is in full flexion, the output of the voltage divider (which is fed into the inverting operational amplifier) is less than the reference voltage thereby dropping the output down linearly to 0 V. Each of the sensors 200 has an associated operational amplifier 362 and in particular, the output from the sensor 200 (i.e., the voltage divider associated therewith) is fed as an input into the operational amplifier 362 associated with the particular sensor 200.

The two outputs from the gain stage 360 (i.e., outputs of the operational amplifier 362) are directed to two A/D ports 322 of the microprocessor 320 (e.g., PIC microprocessor). The illustrated PIC microprocessor 320 has a 10-bit A/D (analog/digital) resolution allowing fine voltage variations to be resolved due to minute bends in the sensors 200. The microprocessor 320 contains an algorithm(s) associated therewith that manipulates and processes the A/D data, and then the PIC microprocessor 320 outputs a signal to activate one of the motors 210 (vibrating element). In one exemplary embodiment, the PIC microprocessor 320 can not supply enough current to drive one motor 210. As a result, the output of the PIC microprocessor 320 is connected to a MOSFET to source approximately 30 mA of current to each motor 210, to thereby drive the motor.

The illustrated circuit in FIG. 9 is constructed so that the bend sensor 200 gives a full 5 V swing for anticipated bending in the anlde brace 100. Since the sensor 200 is simply a variable resistor with values changing only 10-20%, a conventional voltage divider is insufficient for use in the present circuit since such a configuration would only allow a voltage swing of 0.5-1.0 V which is not enough for the present application. In view of the present requirements for the device 100, the operational amplifying circuit is designed to scale and shift the output from the voltage divider to the proper 0-5 V range. In accordance with the present invention, this is accomplished by providing a reference voltage to the operational amplifier 362 with a value between the upper and lower extremes of the voltage outputs from the sensor voltage divider circuit. By increasing the resistance in the current path, any difficulties that may have arose from having a low valued input and feedback resistors for the op amp amplifier are resolved since the use of small valued resistors in place results in the path around the operational amplifier providing a substantial current path causing the voltage at the output of the voltage divider to sag.

The output of the microprocessor 320 is in the form of control signals that are sent to one of the motors 210 to control the operation thereof. For example, when the device 100, and in particular the microprocessor 320, is programmed so that when one of the sensors 200 bends a sufficient degree, this signifies a triggering event as described herein and upon detection of such triggering event, the microprocessor 320 sends a control signal to the respective motor 210 to cause operation (vibration) thereof for a predetermined time period (e.g., at least 0.5 seconds).

As previously mentioned and according to one exemplary embodiment, the device 100 is powered by a plurality of batteries 352, such as three Toshiba coin cell lithium ion batteries, that are connected in series. These batteries 352 are rated at 3 V each and 100 mAh, which when combined in series translates to a 9 V power source. The 9 V rating of the power source is too high for the PIC microprocessor 320, which only requires 5 V, and therefore, the above described voltage regulator circuit is used to regulate down the voltage to 5 V. The regulator 350 (e.g., a 3-Terminal 1 A positive voltage regulator—LM7805) thus converts varying input voltage to a constant 5 V. It can drive current up to 1 A, which is more than is required and can hold up to 36 VDC input voltage.

One purpose of the algorithm(s) associated with the PIC microprocessor 320 is to detect large, abnormal variations in gait, more specifically, ankle roll, and provide feedback to help correct a potential precursor to a fall. The algorithm preferably is capable of auto-calibrating itself to ensure proper functioning of the device 100 in a variety of different operating conditions, such as walking, standing, etc., and to compensate for variability in foot size and geometry. In view of these objects, the algorithm according to one exemplary embodiment has the following features. The inputs to the analog are analog values as read by the sensors 200 and the associated circuitry, while the output are control signals to the motors 210. The PIC microprocessor 320 has a relatively high sampling rate and in particular, the PIC microprocessor 320 preferably samples the values of the sensors 200 at a rate of 100 Hz; however, other rates can be equally used. This allows for subtle and quick abnormalities to be detected by the algorithm (microprocessor).

In order to perform the desired auto-calibration and signal processing, the algorithm stores a recent history of sensor data (e.g., the voltage values from the voltage dividers). The memory that is part of the PIC microprocessor 320 provides the necessary storage area for this history. For example, one exemplary algorithm and PIC 320 combination is constructed so that 13 values can be stored per channel; however, it will be appreciated that this can easily be expanded if the need or desire arises.

The criterion for triggering a control signal to be sent to cause operation of one motor is that the difference between the current sensor value and the average of the history of the values exceeds a predetermined threshold (i.e., the sensor is bent a predetermined extent to trigger operation of the vibrating element). This finite impulse response (FIR) filtering technique, which is also known as a moving-average filter, provides a moving benchmark yielding the desired auto-calibration functionality. This criterion also ensures that stimulation occurs only when the bending is increasing on one given side of the foot. For example, the motor 210 on the lateral side is turned on when the sensor inverts (curvature of the sensor 200 is increasing), but not when the sensor 200 returns to the neutral position as when curvature of the sensor is decreasing.

It will also be appreciated that the microprocessor 320 can be configured so that additional criteria can be implemented to offer the desired functionality. First, a mutually-exclusive stipulation can be added to the motor operation preventing both motors 210 on the device 100 from turning on simultaneously. Vibration to indicate the direction of instability only works properly if the direction is clear to the user. This would not be the case if both motors 210 were allowed to operate at the same time (concurrently). Second, the algorithm can be configured and modified so that the motors 210 are turned on for at least a minimum amount of time, e.g., approximately half a second. This modification ensures that the message of the instability is delivered to and received by the user since too short a vibration might not be perceived by the user.

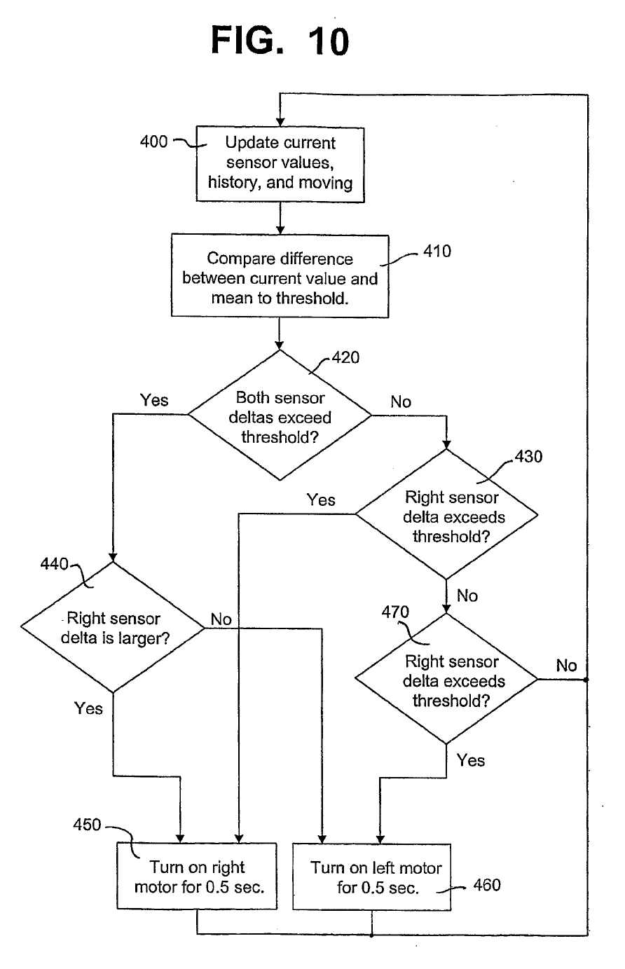

FIG. 10 is a flowchart of the operation of the program associated with the PIC microprocessor 320 according to one exemplary embodiment. In step 400, an update is performed for the current sensor values (e.g., voltage values), history and the moving average of the values (outputted voltage values). At step 410, the difference between the current sensor value and the mean to threshold value is compared. At step 420, it is determined whether both sensor deltas exceed the threshold. If the answer is no, then at step 430, it is determined if the right sensor delta exceeds the threshold. If the answer of step 420 is yes, then at step 440, it is determined whether the right sensor delta is larger and if the answer is yes, then at step 450, the right motor 210 is turned on for at least 0.5 second. If the answer at step 440 is no, then at step 460, the left motor 210 is turned on for at least 0.5 second. If the answer of step 430 is yes in that the right sensor delta exceeds a threshold, then at step 450, the right motor 210 is turned on for at the at least 0.5 second. If the answer of step 430 is no in that the right sensor dealt does not exceed the threshold, then at step 470, it is determined whether the left sensor delta exceeds the threshold. If the answer at step 470 is no, then the program loops back to step 400. If the answer at step 470 is yes, then the left motor 210 is turned on at step 460 for the at least 0.5 second. After operation of either the right motor 210 at step 450 or the left motor 210 at step 470 is operated, then the program loops back to step 400.

It will also be appreciated that the program and operation of the PIC microprocessor 320 can be modified so that algorithmic implementation of filtering is capable of distinguishing between normal, safe ankle roll that occurs during walking and perilous ankle roll that occurs during walking or standing. The algorithm can be configured to recognize a regular pattern in the data as walking and adjust thresholds accordingly so that normal ankle movement that occurs during walking does not trigger the motors 210 in the device 100. Implementation of such functionality requires either an extended history to include an entire gait cycle or an alternative filtering technique, such as the infinite impulse response or an IIR filter, that requires less memory for data storage while still capturing information about the historical average of the data. It will be appreciated that by programming the microprocessor 320 to evaluate a larger time window of data, normal walking action can be distinguished from an ankle roll.

In terms of fabrication of the printed circuit board that contains the above described circuitry, it is desirable to minimize the size and create a fully functional board that is small enough to fit on and be carried by the ankle brace 100. In one embodiment, the PCB is fit into an area with dimensions of 1.5″ by 2″. The largest components include the operational amplifier 362, the microprocessor 320, and the voltage regulator 350, with these components being placed strategically to minimize the length of the copper traces. In a different embodiment, all of the components are constructed on a rectangular PCB that has the dimensions of 1.25″ by 1.6″. However, these dimensions are merely exemplary and, it will be appreciated that the housing can have other dimensions.

A number of quantitative tests were performed to evaluate the performance of the device 100 in alerting the wearer of either inversion of eversion of the ankle. For example, the quantitative tests included force plate testing, and EquiTest testing that is in the form of computerized dynamic posturography that is conducted view NeuroCom's SMART EquiTest machine. In addition, qualitative testing was performed to assess the effectiveness of the device 100. The data from both the quantitative and qualitative tests was very positive since the subjects wearing the device 100 definitely sensed the position of the ankle more effectively when wearing and being assisted by the device 100. The vibratory stimulation was coherent and distinguishable from one side to the other depending on body sway and ankle roll. It was reported that that the ankle brace 100 provided a pleasant feeling of warmth and security, as it was form-fitting and made of a soft material.

The device 100 is preferably constructed so that is satisfies all or substantially all of the following functional and physical requirements. More specifically, the device 100 detects a fall within less than 1 second of fall initiation; prevents the fall with a response time of less than 1 second after fall detection; has an independent power source; effectively assists the user in building confidence in his/her stability; enhances rather than detracts from the user's day to day activities; does not impede the user's gait; is not visually distracting; is durable; is lightweight and portable; is unobtrusive; is comfortable; is cost effective; is easy to deploy/wear; is simple and easy to operate; and does not cause user injury. In addition, the device 100 provides good battery life and employs rechargeable batteries and is universally usable among the elderly.

It will also be understood that while the present invention has been described in terms of its use with elderly patients, the present device 100 is not limited to such use; but instead, the device 100 can be used with other types of patients that are suffering from the same stability issues described herein.

In yet another embodiment, a device according to the present invention is embodied in a structure that is configured to be placed around the wrist of a person that is suffering from carpal tunnel syndrome or a similar type of condition. The device can be similar to the ankle brace described above or it can be different so long as the device is capable of detecting movement of the wrist region and providing movement correction when the individual moves his or her wrist in an undesired, unfavorable manner. More specifically and as mentioned above, the movements of the wrist region of a person suffering from carpal tunnel syndrome must be controlled so that the person does not aggravate or re-injure the problematic area. The present device can be embodied as a wrap that extends around the wrist and includes members, such as sensors, that detect movement, such as bending, of the wrist. The device also includes means for correcting the movement of the individual in response to detection of undesired movement by the individual. In this aspect, the present invention can be thought of as a training device that can assist in controlling and tailoring the behavior of the individual.

For example, if the individual moves his or her wrist in an undesired manner (i.e., in the wrong direction), the sensors, or some other detecting element(s), detect this movement of the wrist as by the sensor bending beyond a threshold amount which results in a controller sending a signal to the means for correcting this movement (e.g., a vibratory or auditory element, etc.). In the case similar to the ankle brace described above, if vibratory elements are used, once the person moves his or her wrist in a manner that causes sufficient bending of the sensors, then the vibratory element can be actuated to cause a short vibration in a local region of the wrist so as to instruct (train) the individual which movement of the wrist is not advisable due to the person's condition, etc., or lack of sense when an injury may be occurring. In this way, the present invention can be used to train an individual which range of movement is appropriate and which range of motion is not appropriate and over time, with the aid of the device, the person will not move the body limb in a direction that is not favorable.

In another aspect, a device according to the present invention can be constructed for placement around the head of a person to alert the person when an undesired condition, such as the head nodding, is occurring, thereby permitting the person to take corrective measures before injury, etc., occurs. As previously mentioned, there is an ever growing problem with individuals falling to sleep at the wheel of a vehicle due to more demanding personal and work schedules and less time for quality sleep. Not only dose lack of sleep put the driver's life and well being in jeopardy but it also jeopardizes the well being of the passengers and other individuals on the road.

The present device can thus be embodied so that it can be placed around the head of the individual and it includes at least one sensor or other means for detecting movement of the head and in particular, for detecting a range of undesired movement for the head once the device is actuated. The device also includes a feedback element for providing feedback to the individual and this feedback can be in the form of a vibration, auditory signal, etc. For example, if the person begins to nod off and the person's head begins to fall, the sensor will be actuated due to movement of the head in a manner that exceeds a threshold extent, such as during a significant head nod. The feedback element can provide a vibration or the like to alert the individual that the range of motion of the head is outside of an acceptable range of motion and awaken the person in the event that the person has fallen asleep.

Moreover, while the present invention has been described above as being in the form of a brace that includes vibratory elements, it will also be appreciated that the brace and the more broadly, the device of the present invention can function in different manners. For example, the present brace has both an active aspect and a passive aspect and it can be used on any number of different parts of the body. The brace is in effect a “smart” device in that it keeps a history of the user's movement and provides feedback based on this time history. The device thus gets to “know” the user's movements so that it can provide feedback if things seem out of the ordinary for the user. The vibratory elements described herein are merely one form of a feedback device that is configured to provide feedback to an individual in direct response to movements by that individual. As mentioned herein, other feedback besides vibratory feedback, including audio or visual or some other type of sensor feedback can be provided to the individual. The present invention is thus not limited to the use of vibratory feedback, e.g., in the head band embodiment, the feedback provided to the individual can be audio (e.g., an alarm) when the individual's head moves in an undesired, excessive manner.

Any number of different types of sensors or detectors can be used in accordance with the present invention including those described above and also, RFID sensors that use radio frequency to communicate between sensing elements and stretch/flex type sensors. The passive element of the device can be made variable or at least the device can be pseudo-passive as in the case where the brace relaxes when the wearer has been inactive for awhile. This element could also serve as an active element, sensing movement or imbalance and modulating the bracing pressure supplied to the user. For example and based on the feedback received from user's movements and the time history of the movements, the pressure exerted by the brace on the user can be varied as when the device recognizes that the user has not been active for a number of minutes, e.g., when sitting or lying down.

The device of the present invention can also provide multiple levels of feedback, e.g., vibration but auditory warning when imbalance is precarious. For example, vibratory feedback can be provided for subtle imbalances or gait changes, while auditory feedback can be provided in the case of more extreme imbalance or impending fall. This would serve the purpose of avoiding the possibility of the user adjusting to the feedback and tuning it out. It can also serve to give both subliminal and fully conscious feedback to the user based on the severity of the imbalance.

In another aspect of the present invention, the device and in particular, the brace of the present invention is an active control brace (active control ankle brace) and the feedback that is provided by the device is in the form of feedback that makes the ankle or other limb do something. The device can be of an implantable type that administers feedback that controls certain nerve movements or the feedback can be in the form of external electric stimulation (actuation); pneumatic/hydraulic movement, controllable foot cushions that cause the foot to tilt or strips or electro-active polymer. In general, the feedback element(s) is in the form of some type of element that can provide feedback to the individual and is not limited to the vibratory feedback disclosed herein.

While the use of batteries as a power supply for the present invention is one preferred way of powering the device, other means exist for powering the device, including, the use of reverse-piezo (voltage generated from stepping powers the circuit); RFID blanket, and a rechargeable power source that operates like a wrist watch that converts motion into power.

It will be appreciated by persons skilled in the art that the present invention is not limited to the embodiments described thus far with reference to the accompanying drawings; rather the present invention is limited only by the following claims.

What is claimed is:

1. A device for detecting and correcting instability in an ankle/foot region of an individual comprising:
a body constructed to be worn around the ankle/foot region of the individual;
first and second sensors arranged so that when the device is worn, the first sensor is positioned at a first location of the foot, the second sensor being positioned at a different second location of the foot;
a controller operatively connected to the first and second sensors;
first and second vibrating elements in communication with the controller, the first vibrating element being located proximate the first sensor, the first vibrating element being actuated for a predetermined time period when the first sensor is bent a first predetermined extent, the second vibrating element being located proximate the second sensor, the second vibrating element being actuated for a predetermined time period when the second sensor is bent a second predetermined extent; and
a power source for powering the device.

2. The device of claim 1, wherein the body comprises an ankle brace that is constructed to fit around the ankle and foot region of the individual and includes fastening elements to ensure the body is securely held in place around the anlde/foot region, the first location being a lateral side of the foot and the second location being a medial side of the foot.

3. The device of claim 1, wherein the first and second sensors and motors are disposed along an inner surface of the body in facing relation to the ankle/foot region and the controller is located along an outer surface of the body.

4. The device of claim 3, wherein the inner surface includes first and second channels formed in the inner surface with the first sensor lying the first channel and the second sensor lying in the second channel, with each of the first and second sensors being covered with a material that extends across and covers the first and second channels so as to space the sensor from the individual.

5. The device of claim 4, wherein the first sensor is positioned for placement near the lateral malleoli of the wearer and the second sensor is positioned for placement near the medial malleoli and the first motor is positioned for placement above the calcaneal fibular ligament underneath the lateral malleolus and the second motor is positioned for placement above the calcaneal fibular ligament underneath the medial malleolus so that the first sensor and first vibrating element alerts the individual to a first undesired movement of the foot and the second sensor and the second vibrating element alerts the individual to a different second undesired movement of the foot.

6. The device of claim 1, wherein each of the first and second sensors detects bending in only one direction.

7. The device of claim 1, wherein each of the first and second sensors comprises a piezo-resistive strip that provides and maintains a change in resistance based on the extent of bending of the strip, the strip being electrically connected to the controller so that when the strip is bent either the first or second predetermined extent, the controller actuates one of the first and second vibrating elements, respectively.

8. The device of claim 1, wherein each of the first and second sensors is constructed so that as the sensor is bent, the resistance thereof gradually increases and flexion of the sensor is converted into a voltage using a voltage divider circuit.

9. The device of claim 8, wherein each of the first and second sensors has an associated operational amplifier which is part of a circuit of the controller into which an output of the voltage divider is fed and which is configured so that when the sensor is not in flexion, the output is 5 V and when the sensor is in full flexion, the output of the voltage divider is dropped down linearly to 0 V.

10. The device of claim 9, wherein each operational amplifier is arranged to have a gain of 7.7 V and a reference voltage of 3.0 V and functions to scale and shift the output from the voltage divider to a 0 to 5 V range by providing a reference voltage to the operational amplifier with a value between maximum and minimum voltage outputs from the voltage divider, wherein an output of each operational amplifier is fed to a pair of A/D (analog/digital) ports on a programmable integrated circuit (PIC) microprocessor that forms part of the controller, the microprocessor process the A/D data and outputs a signal to activate one of the motors.

11. The device of claim 10, wherein the microprocessor includes an auto-calibration feature and is configured to receive analog input values as read by the first and second sensors and the output is a control signal delivered to one motor, the microprocessor sampling the outputs of the voltage dividers associated with the sensors at a predetermined rate and includes memory for storing a history of the outputs of the voltage dividers for the respective sensors.

12. The device of claim 11, wherein the first predetermined extent is when a difference between a current output of the voltage divider associated with the first sensor and an average of the stored history of the outputs of the voltage divider associated with the first sensor exceeds a predetermined threshold; wherein the second predetermined extent is when a difference between a current output of the voltage divider associated with the second sensor and an average of the stored history of the outputs of the voltage divider associated with the second sensor exceeds a predetermined threshold.

13. The device of claim 1, wherein the first predetermined extent is different than the second predetermined extent.

14. The device of claim 1, wherein the first predetermined extent is the same as the second predetermined extent.

15. The device of claim 1, wherein the controller is configured so that only one of the first and second vibrating motors is operated at one time.

16. A device for detecting movement and providing corrective feedback to an individual to influence the movement of the individual comprising:
a first means for detecting movement of the individual;
a controller in communication with the first means and configured to receive and store information relating to the detected movement of the individual over a period of time, the controller defining a range of movements that is considered acceptable based on the detected movements over time and the stored information; and
a second means for providing sensory feedback to the individual when the controller determines that the detected movements lies outside of the acceptable range to assist in correcting the movement of the individual.

17. The device of claim 16, wherein the first means comprises at least one sensor that detects an extent of movement of the individual and the controller stores in memory the extent of movement of the individual over a period of time so as to permit upper and lower limits of the acceptable range of movement to be defined.

18. The device of claim 16, wherein the sensory feedback is at least one feedback selected from the group consisting of physical sensory feedback, visual feedback and auditory feedback.

19. The device of claim 16, wherein the sensory feedback includes a first level of feedback that is actuated when the detected movement lies outside the acceptable range by a first degree and a second level of feedback that is actuated when the detected movement lies outside the acceptable range by a second degree that is greater than the first degree.

20. The device of claim 19, wherein the first degree represents a first amount of imbalance and instability that is detected in an ankle/foot region of the individual and the second degree represents a greater second amount of imbalance and instability that is detected in the ankle/foot region.

21. The device of claim 17, wherein the lower limit and upper limit are adjusted over time as the controller receives the information relating to the detected movement of the individual.

22. The device of claim 16, wherein the device is incorporated in at least one of a structure to monitor and correct head movement; a structure for placement in a wrist region to monitor movement of the wrist, and a structure for placement in an ankle/foot region to monitor movement thereof.

23. A device to detect and correct an undesired movement by an individual comprising:
a body to be placed at a target location of the individual;
at least one sensor arranged so that when the device is worn, the first sensor is positioned at the target location, the sensor being of the type that when a physical state of the sensor is modified, the resistance thereof gradually increases and the extent of the modification of the at least one sensor is converted into a voltage value;
a microprocessor operatively connected to the at least one sensor for receiving and sampling current voltage values outputted from the at least one sensor, the microprocessor including a memory for storing a history of the voltage values outputted from the at least one first sensor;
at least one feedback element in communication with the microprocessor, the at least one feedback element being located proximate the at least one sensor, wherein the microprocessor sends a control signal to operate one of the at least one feedback element when a difference between a current voltage value of the at least one sensor and an average of the stored history of voltages values of the at least one sensor exceeds a predetermined threshold resulting in actuation of the at least one feedback element for a predetermined time period to alert the individual of an occurrence of the undesired movement at the target location; and
a power source for powering the device.

24. A method for detecting and alerting an individual of instability in an ankle/foot region comprising the steps of:
disposing an ankle brace around an ankle and foot region of the individual, the ankle brace including first and second sensors that arranged so that when the device is worn, the first sensor is positioned along one side of the foot, the second sensor being positioned along the other side of the foot, the ankle brace having a first vibrating element located proximate the first sensor and a second vibrating element located proximate the second sensor;
sensing instability in the ankle/foot region when one the first sensor bends a first predetermined extent and the second sensor bends a second predetermined extent due to ankle and foot movement; and
instructing one of the first and second vibrating elements to vibrate for a predetermined amount of time depending upon which of the first and second sensors is bent to the first and second predetermined extents, respectively, to alert the wearer of instability in the ankle/foot region.

25. The method of claim 24, wherein the step of sensing instability comprises the step of:
bending the sensor due to movement of the ankle brace to cause the resistance thereof to gradually increase;
converting flexion of the sensor into a voltage value; and the step of instructing one of the first and second vibrating elements comprises the steps of:
receiving and sampling current voltage values from the sensors;
storing in memory a history of the voltage values from the sensors; and
sending a control signal to operate one of the first and second vibrating elements when a difference between a current voltage value of one sensor and an average of the stored history of voltages values of the one sensor exceeds a predetermined threshold resulting in vibration of the respective vibrating element for the predetermine time period.
